(19) United States
(12) Patent Application Publication
(10) Pub. No.: US 2026/0165919 A1
(43) Pub. Date: Jun. 18, 2026

(54) TOOTHPASTE COMPOSITION

(71) Applicant: Queen Mary University of London, London (GB)

(72) Inventors: Robert Hill, London (GB); Ferranti Wong, London (GB); Florence Wai Ting Mai, London (GB)

(21) Appl. No.: 19/125,136

(22) PCT Filed: Oct. 27, 2023
(86) PCT No.: PCT/EP2023/080106

§ 371 Date: Apr. 28, 2025

(30) Foreign Application Priority Data
Oct. 28, 2022 (GB) 2216019.6
Sep. 6, 2023 (GB) 2313605.4

(51) Int. Cl. A61K 8/21; A61K 8/02; A61K 8/19; A61K 8/365; A61K 8/46; A61K 8/64; A61Q 11/00

(52) U.S. Cl. CPC A61K 8/21; A61K 8/0204; A61K 8/19; A61K 8/365; A61K 8/463; A61K 8/466; A61K 8/64; A61Q 11/00

(57) ABSTRACT

The present invention provides a toothpaste composition comprising fluoride and poly gamma glutamic acid or a salt thereof, a toothpaste tablet and a method of making a toothpaste tablet, comprising the steps of a. providing the toothpaste composition according to any preceding claim; and b. forming the toothpaste composition into a tablet. Furthermore, the invention provides an oral hygiene product comprising the toothpaste composition, a method of treating or preventing dental disease, comprising administering the toothpaste composition, a toothpaste composition or oral hygiene product for use in the treatment or prevention of dental disease and the use of the toothpaste composition or oral hygiene product in the manufacture of a medicament for the treatment or prevention of dental disease.

### TECHNICAL FIELD

The invention relates to oral hygiene compositions, specifically a toothpaste composition. The invention also relates to a method of making a toothpaste composition, an oral hygiene product, a toothpaste tablet, use of said toothpaste composition, oral hygiene product or a toothpaste tablet in the treatment or prevention of dental disease and methods of treating or preventing dental disease. Preferably, the toothpaste composition is a solid toothpaste composition.

### BACKGROUND ART

Dental caries (dental decay) remains the most prevalent and preventable disease in the UK and globally. It is reported that it affects 3.9 billion people (Duangthip, 2022). England alone spends £3.4 billion annually on dental services. A large proportion of this is spent on treating dental caries, using treatment including extractions, fillings, crowns, bridges, dentures and root canal therapies (PHE, 2022). Dental caries occurs when food debris that is not removed from tooth brushing causing a build-up of bacteria in the mouth. This causes an acidic film to form on the enamel surface called ‘dental plaque’. This sticky and acidic biofilm breaks down the surface of the tooth enamel causing demineralisation which can eventually lead to cavitation and/or dental erosion, if not treated (NHSUK, 2018b).

Toothpaste is the most widely used oral care product and is indispensable to the maintenance of good oral health. However, both the packaging and the ingredients can have a huge impact on the environment with the pollution it causes, especially in the ocean (Toothpasteandsociety, 2013). Toothpaste composition ingredients, such as polyvinylpyrrolidone (PVP), when released into our environment have been found to be non-biodegradable, eco-toxic and harmful to aquatic wildlife (Samuelsson, 2014). PVP has been shown to have cytotoxic effects and is harmful to zebra fish (Kizhakkumpat, A et al., 2021, Chae, J et al., 2016). Non-biodegradable PVP is for example used in toothpaste tablets as a binder. This environmental impact is a concern worldwide.

Furthermore, despite advances in oral hygiene, patients worldwide still suffer from dental disease, especially in developing countries and third world countries dental disease is of concern.

Finally, the cost of toothpaste compositions and oral hygiene products is often prohibitive.

Therefore, there is a need for a non-toxic, environmentally friendly, biodegradable, non-cytotoxic, vegan, edible and cost-effective toothpaste composition that is useful for treating or preventing dental disease.

### SUMMARY OF INVENTION

The above objects are achieved by the toothpaste composition comprising fluoride and poly gamma glutamic acid (PgGA) or a salt thereof. The toothpaste compositions described herein are oral hygiene compositions.

Advantageously the toothpaste composition of the present invention may be non-toxic, environmentally friendly, biodegradable, non-cytotoxic, vegan, edible, dental disease treating or preventing and/or cost-effective toothpaste composition.

Surprisingly, it has been found that the toothpaste composition of the invention is 20 times more effective than fluoride alone at reducing demineralisation/tooth decay of tooth-like material (Hydroxyapatite) from 0.02 to 0.0009% reduction in calcium release (see FIG. 4). Furthermore, it was found that in some embodiments of the invention that demineralisation/tooth decay and caries is reduced even further when the PgGA or a salt thereof is present in an amount of from about 0.25 wt % to about 2 wt % in the presence of fluoride.

### Toothpaste Composition

The present invention relates to a toothpaste composition comprising fluoride and poly gamma glutamic acid or a salt thereof.

The toothpaste composition according to the invention may comprise poly gamma glutamic acid as a calcium poly gamma glutamic acid, sodium poly gamma glutamic acid or potassium poly gamma glutamic acid.

The toothpaste composition according to the present invention may comprise the poly gamma glutamic acid or a salt thereof in an amount of from about 0.1 to about 5 wt %, optionally 0.2 to about 2.5 wt %, optionally from about 0.25 wt % to about 2 wt %, optionally from about 0.5 wt % to about 1 wt %.

The toothpaste composition according to the present invention may comprise poly gamma glutamic acid or a salt thereof in an amount of from about 0.25 wt % to about 2 wt % and/or the fluoride from about 500 to about 1700 ppm.

The toothpaste composition according to the present invention may comprise poly gamma glutamic acid or a salt thereof in an amount of from about 0.25 wt % to about 2 wt % and/or the fluoride from about 500 to about 1700 ppm, wherein said poly gamma glutamic acid or a salt is present as solid particles.

The toothpaste composition according to the present invention may comprise poly gamma glutamic acid or a salt thereof in an amount of from about 0.25 wt % to about 2 wt % and/or the fluoride from about 500 to about 1700 ppm, wherein said poly gamma glutamic acid or a salt is present as particles, wherein the particles have a particle size of about 0.1 μm to about 200 μm, or about 10 μm to about 50 μm.

The toothpaste composition according to the present invention may comprise poly gamma glutamic acid or a salt thereof in an amount of from about 0.25 wt % to about 2 wt % and/or the fluoride from about 500 to about 1700 ppm, wherein said poly gamma glutamic acid or a salt is present as particles, wherein the particles have a particle size distribution in which at least about 95% of the particles have a particle size of about 0.1 μm to about 200 μm

The toothpaste composition according to the present invention may comprise poly gamma glutamic acid or a salt thereof in an amount of from about 0.25 wt % to about 2 wt % and/or the fluoride from about 500 to about 1700 ppm, wherein said poly gamma glutamic acid or a salt is present as particles, wherein the particles have a particle size distribution in which at least about 80% of the particles have a particle size of about 10 μm to about 50 μm.

In a preferred embodiments, the toothpaste composition according to the present invention may comprise poly gamma glutamic acid or a salt thereof in an amount of from about 0.25 wt % to about 2 wt % and/or the fluoride from about 500 to about 1700 ppm, wherein said poly gamma glutamic acid or a salt is present as particles, wherein the particles have a particle size distribution in which at least about 95% of the particles have a particle size of about 0.1 μm to about 200 μm, and at least about 80% of the particles have a particle size of about 10 μm to about 50 μm.

Particle size and/or particle size distribution may be measured using any suitable method known to the skilled person. For example, in some embodiments, the particle size and/or particle size distribution may be measured using a light microscope. In some embodiments, the particle size and/or particle size distribution may measured using a light microscope, wherein the particles are in solution (for example in aqueous solution). A suitable microscope may be a Zeiss Universal Polarising Microscope, for example using ImageJ against a graticule imaged with the same optics. Particle size distribution may be determined manually (by eye) or by using a computer program. Alternatively, the particle size and/or particle size distribution may be measured by laser particle size analysis.

The toothpaste composition according to the present invention may comprise poly gamma glutamic acid or a salt thereof in an amount of from about 0.25 wt % to about 2 wt % and/or the fluoride from about 500 to about 1700 ppm, wherein said poly gamma glutamic acid or a salt has a molar mass up for about 800 000 g/mol, for example from about 1000 to about 800 000 g/mol, preferably about 5000 to about 700 000 g/mol.

The toothpaste composition according to the present invention may comprise:
a. Poly gamma glutamic acid or a salt thereof in an amount of from about 0.25 to about 5 wt %;
b. Fluoride in an amount of from about 500 to about 1700 ppm;
c. Sodium bicarbonate in an amount of from about 5 to about 30 wt %;
d. Sodium citrate in an amount of from about 55 to about 85 wt %;
e. Sodium methylcocoyl taurate and/or sodium lauryl sulfate in an amount of from about 0.2 to about 4 wt %;
f. At least an additional component(s) and wherein the additional component(s) is present in an amount of from about 3 to about 15 wt %;
relative to the total weight of the composition.

The toothpaste composition according to the present invention may comprise:
a. Poly gamma glutamic acid or a salt thereof in an amount of from about 0.25 to about 5 wt %;
b. Fluoride in an amount of from about 500 to about 1700 ppm;
c. Sodium bicarbonate in an amount of from about 5 to about 30 wt %;
d. Sodium citrate in an amount of from about 55 to about 85 wt %;
e. Sodium methylcocoyl taurate and/or sodium lauryl sulfate in an amount of from about 0.2 to about 2 wt %;
f. At least an additional component(s) and wherein the additional component(s) is present in an amount of from about 3 to about 15 wt %;
relative to the total weight of the composition.

The toothpaste composition according to the present invention may comprise:
a. Poly gamma glutamic acid or a salt thereof in an amount of from about 0.25 to about 2 wt %;
b. Fluoride in an amount of from about 500 to about 1700 ppm;
c. Sodium bicarbonate in an amount of from about 5 to about 30 wt %;
d. Sodium citrate in an amount of from about 55 to about 85 wt %;
e. Sodium methylcocoyl taurate and/or sodium lauryl sulfate in an amount of from about 0.2 to about 4 wt %;
f. At least an additional component(s) and wherein the additional component(s) is present in an amount of from about 3 to about 15 wt %;
relative to the total weight of the composition.

The toothpaste composition according to the present invention may comprise:
a. Poly gamma glutamic acid or a salt thereof in an amount of from about 0.25 to about 2 wt %;
b. Fluoride in an amount of from about 500 to about 1700 ppm;
c. Sodium bicarbonate in an amount of from about 5 to about 30 wt %;
d. Sodium citrate in an amount of from about 55 to about 85 wt %;
e. Sodium methylcocoyl taurate and/or sodium lauryl sulfate in an amount of from about 0.2 to about 2 wt %;
f. At least an additional component(s) and wherein the additional component(s) is present in an amount of from about 3 to about 15 wt %;
relative to the total weight of the composition.

The toothpaste composition according to the present invention may comprise:
a. Poly gamma glutamic acid or a salt thereof in an amount of from about 0.5 to about 1 wt %;
b. Fluoride in an amount of from about 500 to about 1700 ppm;
c. Sodium bicarbonate in an amount of from about 5 to about 30 wt %;
d. Sodium citrate in an amount of from about 55 to about 85 wt %;
e. Sodium methylcocoyl taurate and/or sodium lauryl sulfate in an amount of from about 0.2 to about 4 wt %;
f. At least an additional component(s) and wherein the additional component(s) is present in an amount of from about 3 to about 15 wt %;
relative to the total weight of the composition.

The toothpaste composition according to the present invention may comprise:
a. Poly gamma glutamic acid or a salt thereof in an amount of from about 0.5 to about 1 wt %;
b. Fluoride in an amount of from about 500 to about 1700 ppm;
c. Sodium bicarbonate in an amount of from about 5 to about 30 wt %;
d. Sodium citrate in an amount of from about 55 to about 85 wt %;
e. Sodium methylcocoyl taurate and/or sodium lauryl sulfate in an amount of from about 0.2 to about 2 wt %;
f. At least an additional component(s) and wherein the additional component(s) is present in an amount of from about 3 to about 15 wt %;
relative to the total weight of the composition.

The toothpaste composition according to the present invention may comprise poly gamma glutamic acid or a salt thereof having a molar mass from about 1000 to about 800 000 g/mol, preferably about 5000 to about 700 000 g/mol.

The toothpaste composition according to the present invention may comprise poly gamma glutamic acid or a salt thereof in a free form, preferably in a free form within a particle.

The toothpaste composition according to the present invention may comprise poly gamma glutamic acid or a salt thereof in a particulate form.

The toothpaste composition according to the present invention may comprise poly gamma glutamic acid or a salt thereof in particulate form with a particle size of about 0.1 μm to about 200 μm, or about 10 μm to about 50 μm.

The toothpaste composition according to the present invention may comprise poly gamma glutamic acid or a salt thereof in particulate form with a particle size distribution in which at least about 95% of the particles are in the range of about 0.1 μm to about 200 μm.

The toothpaste composition according to the present invention may comprise poly gamma glutamic acid or a salt thereof in particulate form with a particle size distribution in which at least about 80% of the particles are in the range of about 10 μm to about 50 μm.

Preferably, the toothpaste composition according to the present invention may comprise poly gamma glutamic acid or a salt thereof in particulate form with a particle size distribution in which at least about 95% of the particles are in the range of about 0.1 μm to about 200 μm, and about 80% of the particles are in the range of about 10 μm to about 50 μm.

Particle size and/or particle size distribution may be measured using any suitable method known to the skilled person. For example, in some embodiments, the particle size and/or particle size distribution may be measured using a light microscope. In some embodiments, the particle size and/or particle size distribution may measured using a light microscope, wherein the particles are in solution (for example in aqueous solution). A suitable microscope may be a Zeiss Universal Polarising Microscope, for example using ImageJ against a graticule imaged with the same optics. Particle size distribution may be determined manually (by eye) or by using a computer program. Alternatively, the particle size and/or particle size distribution may be measured by laser particle size analysis.

The toothpaste composition according to the present invention may comprise poly gamma glutamic acid or a salt thereof selected from the group consisting of: a mixture of D and L stereo isomers of glutamic acid, D isomer of glutamic acid and L isomer of glutamic acid. However, the stereo isomer that is used is not important, since either stereoisomer or a mixture of the two stereoisomers can achieve the advantages of the invention.

The toothpaste composition according to the present invention may comprise fluoride in an amount of from about 100 to about 10000 ppm, optionally 200 to about 3000 ppm, optionally from about 500 to about 1700 ppm.

The toothpaste composition according to the present invention may comprise fluoride selected from the group consisting of: fluoride-containing bioactive glass, fluoride-containing alkalisite glass, sodium fluoride, potassium fluoride, sodium fluoride and sodium monofluorophosphate.

The toothpaste composition according to the present invention may further comprise sodium bicarbonate, potassium bicarbonate, calcium carbonate or a mixture thereof.

The toothpaste composition according to the present invention may further comprise sodium bicarbonate.

The toothpaste composition according to the present invention may further comprise sodium bicarbonate, potassium bicarbonate, calcium carbonate or a mixture thereof in an amount of from about 5 to about 30 wt %

The toothpaste composition according to the present invention may further comprise sodium bicarbonate in an amount of from about 5 to about 30 wt %.

The toothpaste composition according to the present invention may further comprise sodium bicarbonate in an amount of from about 15 to about 25 wt %.

The toothpaste composition according to the present invention may further comprise sodium citrate, casein phosphopeptide, potassium nitrate or a mixture thereof.

The toothpaste composition according to the present invention may further comprise sodium citrate, casein phosphopeptide, potassium nitrate or a mixture thereof in an amount of from about 55 to about 85 wt %.

The toothpaste composition according to the present invention may further comprise sodium citrate in an amount of from about 55 to about 85 wt %.

The toothpaste composition according to the present invention may further comprise sodium citrate in an amount of from about 68 to about 75 wt %.

The toothpaste composition according to the present invention may further comprise sodium methylcocoyl taurate, sodium lauryl sulfate, sodium lauryl sulfoacetate (SLSA), sodium cocoyl glycinate, disodium sodium cocoyl glutamate, sodium cocoyl glutamate, decyl glucoside, lauryl glucoside, betaine or a mixture thereof.

The toothpaste composition according to the present invention may further comprise methylcocoyl taurate, sodium lauryl sulfate, sodium lauryl sulfoacetate (SLSA), sodium cocoyl glycinate, disodium sodium cocoyl glutamate, sodium cocoyl glutamate, decyl glucoside, lauryl glucoside, betaine or a mixture thereof in an amount of from about 0.2 to about 4 wt %.

The toothpaste composition according to the present invention may further comprise sodium methylcocoyl taurate, sodium lauryl sulfate in an amount of from about 0.2 to about 4 wt %.

The toothpaste composition according to the present invention may further comprise one or more additional component(s).

The toothpaste composition according to the present invention may further comprise one or more additional component(s) selected from the group of flavourings, colorants, odorants and/or texturizers.

The toothpaste composition according to the present invention may further comprise the additional component(s) in an amount of from about 3 to about 15 wt %.

The toothpaste composition according to the present invention may further comprise the additional component(s) in an amount of from about 6.25 to about 9.5 wt %.

The toothpaste composition according to the present invention may further comprise one or more additional component(s) selected from the group of sorbitol, CaCO₃, kaolin, Hydrated silica, yeast extract, menthol, Mentha arvensis leaf oil, stevioside, Mentha piperita oil, eucalyptus, titanium dioxide and aroma.

The toothpaste composition according to the present invention may be a powder, paste, tablet or capsule. The toothpaste composition according to the present invention may be a dissolvable powder, paste, tablet or capsule. In some embodiments, the toothpaste composition may be in the form of a dissolvable tablet.

Preferably the toothpaste composition of the invention is solid. In more preferred embodiment, therefore, the toothpaste composition is a dissolvable solid tablet composition.

A toothpaste tablet is a tablet that is crunched or chewed during use. The toothpaste tablets are therefore chewable solid table compositions. Toothpaste tablets are different from buccal tablets, which are generally not considered oral hygiene products since buccal tablets are used to deliver an active pharmaceutical ingredient, and additionally are not crunched or chewed. By contrast, the toothpaste compositions (in particular the toothpaste compositions in the form of dissolvable solid table compositions) are oral hygiene products. Therefore, the solid toothpaste tablet composition is not a buccal tablet.

### Poly Gamma Glutamic Acid (PgGA)

Poly-gamma glutamic acid (PgGA) is a biopolymer, a polypeptide synthesized by water and soil bacteria, such as Bacillus subtilis (natto) and B. licheniformis during fermentation. These bacteria can be isolated from fermented soybeans known as Natto in Japan. PgGA is made up of repeating units of L-glutamic acid, D-glutamic acid or both linked through the side chain carboxylic acid rather than the alpha carboxylic acid group. As a completely biodegradable and it has a low pollutant output and is better for the environment, both to manufacture and as an ingredient.

It is an edible biopolymer that occurs naturally in fermented food products. These products include fermented soybeans (Natto in Japanese cuisine) and fermented maize/millet (Poto poto/degue in African cuisine) which are all associated with Bacillus subtilis. The production of PgGA by the bacteria in these fermented products gives it is characteristic sticky and chewy texture (Ajayeoba T A, 2019).

PgGA has many medical applications, including pharmaceutical and biomedical applications. It has been used as a drug carrier, in tissue engineering systems, in stimulating and improving immune activity and in regenerative medicine.

PgGA is water soluble and has adhesive properties.

PgGA has a high molecular weight which affects its efficacy in industrial application which is why techniques have been derived to reduce its molecular weight without destroying its chemical structure (Ogunleye, 2015).

The structure of PgGA, is composed of D- and L-glutamic acid monomers, joined by amide linkages between the amino and gamma carboxyl group. It has a chiral centre in the centre of each unit therefore is optically active. It is anionic and has a negative charge. The structure is highlighted in FIGS. 1 and 2 (Najar and Das, 2019).

PgGA forms 3 types of active stereo isomers: the polymer of D-Glutamate (D-PGA), the polymer of L-Glutamate (L-PGA), and the copolymer composed of D- and L-Glutamate (D-L-PGA) (Najar and Das, 2019). The glutamic acid is polymerised to form a gamma-peptide bond. This bond cannot be acid hydrolysed by alpha proteases, but it can be hydrolysed by several bacterial species in the gut. Only gamma-glutamyl transpeptidase, produced by bacteria can hydrolyse PgGA. The alpha-peptide bond found in most proteins can be by readily hydrolysed by most proteases, therefore can be hydrolysed in the oral environment. However, gamma-poly glutamic acid is not easily broken down in the oral environment (Qamar et al., 2016).

The major building block of PgGA is the glutamate residue which has 3 functional groups: alpha-NH₂ alpha-COOH and alpha-COOH. In chemically catalysed polymerisation alpha-peptide bonds form between alpha-NH₂ and alpha-COOH forming alpha-polyglutamic acid. During the fermentation polymerisation process L-Glutamate can be converted to D-glutamate which is less degradable by enzymes. This then is copolymerised via the formation of gamma-peptide bonds to form of gamma-(D,L)-PGA or gamma-(D,L)-poly glutamic acid (Qamar et al., 2016).

It is synthesised inside the cell membrane and forms an extracellular capsule. Unlike most proteins, its production does not involve ribosomes and it is not genetically encoded. PgGA is slightly soluble free acid form (with H⁺) or very soluble salt form (with Na⁺, Mg²⁺, K⁺, NH⁴⁺ or Ca²⁺) (Ogunleye et al., 2015).

PgGA is typically used in alcohol free mouthwash at 1000 ppm. PgGA is in solution in wet toothpastes, rinses and mouthwashes; rather than being present in a solid particulate form, as in the present invention.

Frequently, PgGA is complexed with other wet toothpaste, rinse or mouthwash components. For example, PgGA is frequently complexed with amorphous calcium phosphate (ACP). ACP is typically complexed with milk proteins, casein or PgGA. The casein or PgGA is used to stabilize the ACP, while the ACP is used as a source of calcium and phosphate ions. These complexes are not used for the treatment of or the prevention of dental caries. The presence of ACP together with fluoride leads to the formation of a chemically stable fluorapatite (ACPF), which results in no free source of fluoride being available. Further, as the fluoride stabilises the ACP this prevents the calcium from being available (see U.S. Pat. Nos. 9,295,628 B2, 8,673,363 B2, 7,312,193 B2 and CHEN, X, 2017). The PgGA used in the present invention is not complexed to another component, and is used in free form.

PgGA is a cost effective, naturally produced polymer that can be produced renewably from bacteria isolated from fermented soybeans. Its production involves high-viscosity fermentation and the optimum requirements for fermentation has been studied intensively. Large-scale application has proved to be a problem—the highest volumetric concentration of PgGA being lower than 85 g/L. During fermentation it is imperative to control several elements within the processing namely: carbon, nitrogen, metal ions, temperature, aeration, and pH which can all affect PgGA's yield.

Research currently is focusing on optimising the growth conditions to produce high yield, looking at optimal enantiomeric compositions and molecular weight of PgGA to reduce cost (Ogunleye, 2015). The substrate cost is relatively low, examples of inexpensive substrates are molasses (sugar cane refining by-product) and rice straw. The bacteria itself can be easily isolated from fermented soybeans which are also relatively inexpensive and biodegradable (Wang et al., 2020).

The poly gamma glutamic acid may be present as a calcium poly gamma glutamic acid, sodium poly gamma glutamic acid or potassium poly gamma glutamic acid.

The poly gamma glutamic acid or salt thereof may be present in an amount of from about 0.1 to about 5 wt %, optionally 0.2 to about 2.5 wt %, optionally from about 0.25 wt % to about 2 wt %, optionally from about 0.5 wt % to about 1 wt %.

The poly gamma glutamic acid or salt thereof may have a molar mass from about 1000 to about 800 000 g/mol, preferably about 5000 to about 700 000 g/mol.

The poly gamma glutamic acid may be selected from the group consisting of: a mixture of D and L stereo isomers of glutamic acid, D isomer of glutamic acid and L isomer of glutamic acid. However, the stereo isomer that is used is not important, since either stereoisomer or a mixture of the two stereoisomers can achieved the advantages of the invention.

The poly gamma glutamic acid or salt thereof may act as a binder. A binder is generally an agent that prevents the separation of solid or liquid components. The binder may hold the tablet together. The binder may be a thickening agent.

The poly gamma glutamic acid may be of bacterial origin.

The poly gamma glutamic acid may be of Bacillus subtilis origin.

The poly gamma glutamic acid may be a mixture of D and L stereo isomers, for example in a ratio of about 50:50.

The poly gamma glutamic acid may be present in a free form, preferably in a free form within a particle.

The poly gamma glutamic acid may be present in a particulate form, such as a particle.

The poly gamma glutamic acid may be present in particulate form with a particle size of about 0.1 μm to about 200 μm, or about 10 μm to about 50 μm.

The poly gamma glutamic acid may be present in particulate form wherein the particle size distribution is at least 95% of the particles have a size of about 0.1 μm to about 200 μm.

The poly gamma glutamic acid may be present in particulate form wherein the particle size distribution is at least 80% of the particles have a size of about 10 μm to about 50 μm.

Preferably, poly gamma glutamic acid may be present in particulate form wherein the particle size distribution is at least about 95% of the particles have a size of about 0.1 μm to about 200 μm, and about 80% of the particles have a size of about 10 μm to about 50 μm.

Particle size and/or particle size distribution may be measured using any suitable method known to the skilled person. For example, in some embodiments, the particle size and/or particle size distribution may be measured using a light microscope. In some embodiments, the particle size and/or particle size distribution may measured using a light microscope, wherein the particles are in solution (for example in aqueous solution). A suitable microscope may be a Zeiss Universal Polarising Microscope, for example using ImageJ against a graticule imaged with the same optics. Particle size distribution may be determined manually (by eye) or by using a computer program. Alternatively, the particle size and/or particle size distribution may be measured by laser particle size analysis.

Other methods for assessing the particle size distribution may be static laser light scattering under aqueous media.

A small particle size, as used herein, may allow the PgGA to enter into solution (saliva) more easily, when administered, thereby improving the beneficial effects of the toothpaste compositions to a surprising degree, including demonstrating a synergistic improvement with the fluoride.

The poly gamma glutamic acid is preferably present in a solid form.

The poly gamma glutamic acid may not be present in a complexed form. In particular, the poly gamma glutamic acid is not in a poly gamma glutamic acid and amorphous calcium phosphate complex.

### Sodium Fluoride

Fluoride containing toothpaste is universally the best method to prevent dental caries. Fluoride toothpaste should be used twice a day, brushing for two minutes (NHSUK, 2018a). Fluoride aids remineralisation of the enamel surface. It works by converting hydroxyapatite to more acid-resistant fluorapatite during remineralisation. Remineralisation is reliant on calcium and phosphate ions being present to reconstruct the enamel after demineralisation—in a non-cavitated lesion (Featherstone, 2008). Additionally, fluoride inhibits demineralisation at the enamel surface and also inhibits bacterial enzymes involved in the decay process (JD, 1999).

However, fluoride has its limitations. For example, there are concerns about fluoride's safety and toxicity. Nevertheless, the benefits of fluoride outweigh its risks and costs.

The fluoride may be present in an amount of from about 100 to about 10000 ppm, optionally 200 to about 3000 ppm, optionally from about 500 to about 1700 ppm.

The fluoride may be selected from the group consisting of: fluoride-containing bioactive glass, fluoride-containing alkalisite glass, sodium fluoride, potassium fluoride, sodium fluoride and sodium monofluorophosphate.

A biologically active (or bioactive) material is one which, when implanted into living tissue, induces formation of an interfacial bond between the material and the surrounding tissue. Bioactive glasses are a group of surface-reactive glasses, which exhibit bioactivity. The bioactivity of these glasses is the result of complex reactions which take place on the surface of the glass under physiological conditions, and which result in the formation of hydroxycarbonated apatite (HCA) on the surface of the glass. The term “bioactive glass” as used herein is intended to encompass bioactive glass-ceramics as well as bioactive glasses. Bioactive glass-ceramics are similar to bioactive glasses but contain a crystalline phase in addition to the glass phase.

The fluoride-containing bioactive glass may comprise SiO₂, P₂O₅ and a fluoride.

EP2585409B1 details further fluoride-containing bioactive glass that may be used. EP2585409B1 is hereby incorporated by reference.

Although fluoride is generally included in toothpaste compositions, the inventors have surprisingly found that the combination of fluoride and poly gamma glutamic acid or a salt thereof provides superior toothpaste composition compared to the prior art.

### Sodium Bicarbonate

The toothpaste composition according to the present invention may further comprise sodium bicarbonate, potassium bicarbonate, calcium carbonate or a mixture thereof.

The toothpaste composition according to the present invention may further comprise sodium bicarbonate, potassium bicarbonate, calcium carbonate or a mixture thereof in an amount of from about 5 to about 30 wt %.

The toothpaste composition according to the present invention may further comprise sodium bicarbonate in an amount of from about 5 to about 30 wt %.

The toothpaste composition according to the present invention may further comprise sodium bicarbonate in an amount of from about 15 to about 25 wt %.

### Sodium Citrate

The toothpaste composition according to the present invention may further comprise sodium citrate, casein phosphopeptide, potassium nitrate or a mixture thereof.

The toothpaste composition according to the present invention may further comprise sodium citrate, casein phosphopeptide, potassium nitrate or a mixture thereof in an amount of from about 55 to about 85 wt %.

The toothpaste composition according to the present invention may further comprise sodium citrate in an amount of from about 68 to about 75 wt %.

The toothpaste composition according to the present invention may further comprise sodium citrate in an amount of from about 55 to about 85 wt %.

### Sodium Methylcocoyl Taurate/Sodium Lauryl Sulfate 1 wt %

The toothpaste composition according to the present invention may further comprise sodium methylcocoyl taurate, sodium lauryl sulfate, sodium lauryl sulfoacetate (SLSA), sodium cocoyl glycinate, disodium sodium cocoyl glutamate, sodium cocoyl glutamate, decyl glucoside, lauryl glucoside, betaine or a mixture thereof.

The toothpaste composition according to the present invention may further comprise methylcocoyl taurate, sodium lauryl sulfate, sodium lauryl sulfoacetate (SLSA), sodium cocoyl glycinate, disodium sodium cocoyl glutamate, sodium cocoyl glutamate, decyl glucoside, lauryl glucoside, betaine or a mixture thereof in an amount of from about 0.2 to about 4 wt %.

The toothpaste composition according to the present invention may further comprise sodium methylcocoyl taurate or sodium lauryl sulfate in an amount of from about 0.2 to about 4 wt %.

The toothpaste composition according to the present invention may further comprise sodium methylcocoyl taurate or sodium lauryl sulfate in an amount of from about 0.2 to about 2 wt %.

### Additive

The toothpaste composition according to the present invention may further comprise one or more additional component(s).

The toothpaste composition according to the present invention may further comprise one or more additional component(s) selected from the group of flavourings, colorants, odorants and texturizer.

The toothpaste composition according to the present invention may further the additional component(s) in an amount of in an amount of from about 3 to about 15 wt %.

The toothpaste composition according to the present invention may further the additional component(s) in an amount of in an amount of from about 6.25 to about 9.5 wt %.

The toothpaste composition according to the present invention may further comprise one or more additional component(s) selected from the group of sorbitol, CaCO₃, kaolin, Hydrated silica, yeast extract, menthol, Mentha arvensis leaf oil, stevioside, Mentha piperita oil, eucalyptus, titanium dioxide and aroma.

### Tablets or Capsules

The toothpaste composition according to the present invention may be a powder, paste, tablet or capsule. In some embodiments the toothpaste composition is in the form of a powder, paste, tablet or capsule, wherein said powder, paste, tablet or capsule are in solid form. In some embodiments the toothpaste composition is in the form of a solid tablet, preferably a dissolvable solid tablet, still more preferably a chewable dissolvable solid tablet.

The toothpaste composition may be dissolvable. For example, in some embodiments, the toothpaste composition according to the present invention may be a dissolvable powder, paste, tablet or capsule.

The toothpaste composition may be chewable.

The toothpaste composition is generally not in the form of a buccal tablet.

The toothpaste tablet may be obtained or obtainable according to a method of the present invention.

In some embodiments the toothpaste composition is in the form of a dissolvable tablet, preferably a dissolvable solid tablet, still more preferably a chewable dissolvable solid tablet.

In one embodiment, there is provided a toothpaste composition that is an oral hygiene product in the form of a dissolvable, chewable solid tablet composition, comprising:
a. Poly gamma glutamic acid or a salt thereof in free particulate form in an amount of from about 0.5 to about 1 wt %;
b. Fluoride in an amount of from about 500 to about 1700 ppm;
c. Sodium bicarbonate in an amount of from about 5 to about 30 wt %;
d. Sodium citrate in an amount of from about 55 to about 85 wt %;
e. Sodium methylcocoyl taurate and/or sodium lauryl sulfate in an amount of from about 0.2 to about 2 wt %;
f. At least an additional component(s) and wherein the additional component(s) is present in an amount of from about 3 to about 15 wt %;
relative to the total weight of the composition.

In one embodiment, there is provided a toothpaste composition that is an oral hygiene product in the form of a dissolvable, chewable solid tablet composition, comprising:
a. Poly gamma glutamic acid or a salt thereof in free particulate form in an amount of from about 0.5 to about 1 wt %, in which at least 95% of the particles have a size range of from about 0.1 microns to about 200 microns, and optionally in which at least 80% of the particles have a size range of from about 10 microns to about 50 microns;
b. Fluoride in an amount of from about 500 to about 1700 ppm;
c. Sodium bicarbonate in an amount of from about 5 to about 30 wt %;
d. Sodium citrate in an amount of from about 55 to about 85 wt %;
e. Sodium methylcocoyl taurate and/or sodium lauryl sulfate in an amount of from about 0.2 to about 2 wt %;
f. At least an additional component(s) and wherein the additional component(s) is present in an amount of from about 3 to about 15 wt %;
relative to the total weight of the composition.

### Method of Making Toothpaste Composition

The present also provides a method of making the toothpaste composition comprising the steps of:

a. Admixing the components.

The method of making the toothpaste composition may comprise the steps of:
a. providing the toothpaste composition components;
b. mixing said toothpaste composition components, optionally in a tumbler mixer, to obtain a mixed toothpaste composition;
c. optionally sieving said mixed toothpaste composition to obtain a toothpaste composition;
optionally through a fine mesh sieve.

### Method of Making Toothpaste Tablet

The present invention also provides a method of making toothpaste tablet comprising the steps of:
a. providing the toothpaste composition or toothpaste composition components according to the invention; and
b. forming the toothpaste composition into a tablet.

The method of making toothpaste tablet may comprise the steps of:
a. providing the toothpaste composition or toothpaste composition components according to the present invention, optionally in an amount of from about 0.1 to about 5 g; and
b. forming the toothpaste composition into a tablet.

The method of making toothpaste tablet may comprise the steps of:
a. adding the toothpaste composition or toothpaste composition components according to the present invention, to a die, optionally wherein from about 0.1 to about 5 g of the toothpaste composition of the present invention is added to the die;
b. pressing said toothpaste composition within the die to form a tablet.

In some embodiments, step b is performed at a pressure of from about 500 to about 1000 psi. optionally from about 650 to about 850 psi

In some embodiments, the amount of toothpaste composition that is added to the die is from about 0.5 to about 5 g.

### Oral Hygiene Product

The present invention also provides oral hygiene products incorporating the toothpaste composition of the invention. An oral hygiene product is a product that protects against periodontal or cariogenic bacteria and associated dental diseases, such as dental caries.

There is therefore provided an oral hygiene product comprising the toothpaste composition according to the present invention. An oral hygiene product may be, for example, a paste, powder, tablet, a capsule, or a container comprising the toothpaste composition of the invention. In some embodiments, the oral hygiene product is a solid tablet, for example a dissolvable solid tablet, preferably a chewable dissolvable solid tablet.

### Therapy

The present invention provides methods of treating or preventing dental disease using the toothpaste composition according to present invention.

A method of treating or preventing dental disease may comprise administering the toothpaste composition according to the present invention, the toothpaste tablet of the present invention or the oral hygiene product the present invention to a subject.

Also disclosed herein is a method of treating or preventing dental disease in a subject comprising administering to said subject an effective amount of the toothpaste composition according to the present invention, the toothpaste tablet of the present invention or the oral hygiene product the present invention

The present invention also provides a toothpaste composition according to the present invention for use in medicine, for example for use in the treatment or prevention of dental disease.

The present invention also provides a tablet of the present invention or oral hygiene product according to the present invention for use in medicine, for example for use in the treatment or prevention of dental disease.

The present invention also provides use of the toothpaste composition according to the present invention or toothpaste table of the present invention or oral hygiene product according to the present invention in the manufacture of a medicament for the treatment or prevention of dental disease.

The present invention also provides use of the toothpaste composition according to the present invention or toothpaste table of the present invention or oral hygiene product according to the present invention in cleaning teeth.

Dental disease may be selected from the group of periodontal bacterial diseases, cariogenic bacterial diseases, periodontal disease, dental disease and dental caries.

It is noted that the invention relates to all possible combinations of features described herein, preferred in particular are those combinations of features that are present in the claims. It will therefore, be appreciated that all combinations of features relating to the composition according to the invention; all combinations of features relating to the method according to the invention and all combinations of features relating to the composition according to the invention and features relating to the method according to the invention are described herein.

It is further noted that the terms “including”, “comprising”, “having”, “containing” or “involving” do not exclude the presence of other elements. However, it is also to be understood that a description on a product/composition comprising certain components also discloses a product/composition consisting of these components. The product/composition consisting of these components may be advantageous in that it offers a simpler, more economical method for the preparation of the product/composition.

Similarly, it is also to be understood that a description on a method comprising certain steps also discloses a method consisting of these steps. The method consisting of these steps may be advantageous in that it offers a simpler, more economical method.

When values are mentioned for a lower limit and an upper limit for a parameter, ranges made by the combinations of the values of the lower limit and the values of the upper limit are also understood to be disclosed.

### FIGURES DESCRIPTION

FIG. 1: Structural formula of PgGA

FIG. 2: Ball and stick model of PgGA

FIG. 3: ISE experiments were conducted from 3 hours on varnished HAP discs that had been treated with toothpaste tablets of different formulations for 2 minutes before immersion into 0.1M acetic acid solution at 37° C. Combining PgGA with fluoride surprisingly results in a synergistic reduction in demineralisation in the lab.

FIG. 4: ISE result showing Ca²⁺ release from HAP discs (20% porosity) in 50 ml 0.1M acetic acid solution, pH4, for after 3 hours being treated with toothpaste tablets of different formulations for 2 minutes before immersion in a bar chart. Combining PgGA with fluoride surprisingly results in a synergistic reduction in demineralisation in the lab.

FIG. 5: HAP powder was used to increase the surface area for potential binding of PgGA. The dark grey line indicates HAP powder under the FTIR machine. Magnified area of FTIR spectra of PgGA powder, HAP powder and HAP powder treated with our 2% PgGA toothpaste tab in the region wavelength 1100 to 1300. The light grey line indicates our PgGA powder only under the FTIR machine in FIG. 5. The black line indicates our HAP powder treated with our toothpaste tab of 2% PgGA and rinsed. The results from the fluoride and non-fluoride results were the same so, only one of the graphs is shown. We can see a correlation between these lines at 1219 cm⁻¹ indicative of the C—O bond.

FIG. 6: HAP powder was used to increase the surface area for potential binding of PgGA. The dark grey line indicates HAP powder under the FTIR machine. Magnified area of FTIR spectra of PgGA powder, HAP powder and HAP powder treated with our 2% PgGA toothpaste tab in the region wavelength 1600 to 1800. the moderate grey line indicated our PgGA powder only under the FTIR machine. The light grey line indicated our HAP powder treated with our toothpaste tab of 2% PgGA and rinsed. The results from the fluoride and non-fluoride results were the same so, only one of the graphs is shown. We can see a correlation between these lines at 1734 cm⁻¹ indicative of the C═O bond.

FIG. 7: Light microscope images of the PGGA particles (a), b) and c)). Such images were used to measure the particle size distribution. The whole scale is 10 μm.

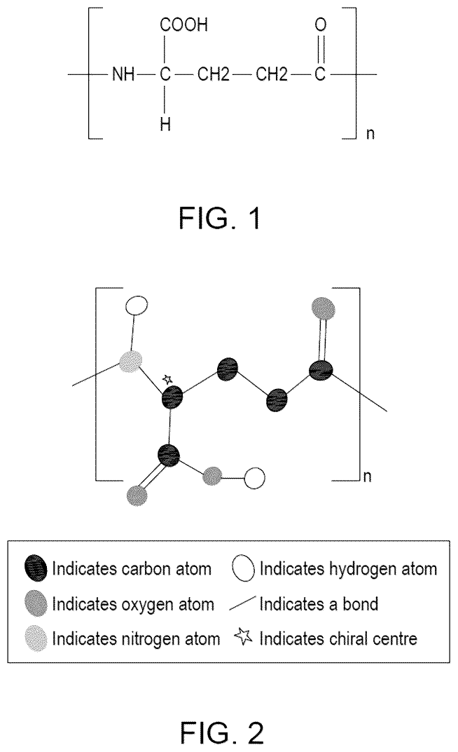

### EXPERIMENT

### Toothpaste Composition

The concentration of PgGA was varied but the concentration of fluoride remained constant at 1450 ppm which equated to 0.32% of our individual tablet formulation. The PgGA was solid and in the form of particles. The particle size was determined using light microscopy. 40 g of each concentration was made. This allowed the ingredients to be weighed more accurately for formulation. The table below shows the grams of ingredients in each 40 g container of toothpaste compositions to formulate the concentrations (0%, 0.5%, 1.0%, 0% with fluoride, 0.5% with fluoride and 1.0% with fluoride). Furthermore, no other polymer binder was present except from PgGA.

| | | | | Sodium lauryl | Sodium | Sodium | Sodium |
|---|---|---|---|---|---|---|---|
| | PgGA | Fluoride | PgGA | sulfate SLS | bicarbonate | fluoride NaF | citrate |
| Example | (wt %) | ppm | (g) | (g) | (g) | (g) | (g) |
| Ex 1 | 0 | 1450 | 0 | 0.4 | 8 | 0.128 | 31.472 |
| Ex 2 | 0.5 | 1450 | 0.2 | 0.4 | 8 | 0.128 | 31.272 |
| Ex 3 | 1 | 1450 | 0.4 | 0.4 | 8 | 0.128 | 31.072 |
| Ex 4 | 0 | | 0 | 0.4 | 8 | 0 | 31.6 |
| Ex 5 | 0.5 | | 0.2 | 0.4 | 8 | 0 | 31.4 |
| Ex 6 | 1 | | 0.4 | 0.4 | 8 | 0 | 31.2 |

### Fabrication of the Toothpaste Tablets

The toothpaste composition of six different concentrations (0%, 0.25%, 0.5%, 1.0%, 1.5%, 2%) was pressed into toothpaste tablets. A minimum of three toothpaste tablets of each concentration was produced to allow repeat testing for the HAP discs.

A hydraulic press machine was used to produce the toothpaste tablets with a disc/cylindrical shaped die. 0.04 oz (1 g) of powder ingredients was placed in the die, which was inserted into the hydraulic press machine and pressed into a tablet form. This was pressed at the pressure 750 psi which then formed a disc shaped tablet.

### HAP Discs

HAP discs act as a model reference material due to the fact it is more consistent and more reproducible than human enamel which contains a variable content and strongly influences its acid dissolution behaviour. An alternative to HAP discs may be enamel from extracted unerupted wisdom teeth (that have not been exposed to fluoride).

The HAP discs of 20% porosity (obtained from Plasma Biotal Matlock UK) were coated in nail varnish leaving only one side unvarnished to ensure the surface area is constant in all our experiments.

### Calibration of Ca²⁺ Electrode

Five standard calcium solutions were prepared according to the manufacturer's instructions were used to calibrate the calcium ion selective electrode used in the study. This included 0.1, 1.0, 10, 100, and 1000 ppm solutions. These were made by serial dilution with the 0.1M Acetic acid, pH4. The Ca²⁺ ISE was paired with a double-junction lithium acetate reference electrode. The calibration occurred at 37° C. (+/−1.0° C.) while using a magnetic stirrer. The electrode is calibrated using the standard calcium solutions stated and a calibration curve is developed to check sensitivity. The calibration curve was developed by plotting the logarithm of calcium activity in millimoles (mmol) against the ISE readings in millivolts (MV). ELIT software computer programme was used to record the data from the ISE experiment.

### Fourier-Transform Infrared (FTIR) Spectroscopy

This analytical technique was used to obtain an infrared spectrum of absorption of our sample, to identify its components. The goal was to obtain information as to whether PgGA bind to the hydroxyapatite, to show its mechanism of action. FTIR was carried out using equipment and software from ‘Perkin Elmer—IR System Spectrum GX. Scans were set to the range 500-2000 cm⁻¹ in wavelength. To ensure the stage was clean, ethanol was used as a cleaning agent to remove anything that would affect the reading.

A 2% PgGA toothpaste tab with fluoride and a 2% PgGA toothpaste tab without fluoride was dissolved in two different containers with 20 ml of deionised water. 5 g of HAP powder was then added to each container. Both containers where then centrifuged for 6 minutes at 4000 speed. The excess liquid was then taken off both tubes with a pipette. 20 ml of deionised water was further added into the containers to rinse of any excess PgGA. This was then centrifuged for a further 4 minutes at 4000 speed. The excess liquid was removed and both powders were allowed to dry overnight in an 37 c oven. This powder was then scanned under FTIR. A reference scan was used that was HAP powder only (disc powdered up).

### Demineralising Solution Preparation

Demineralising solution of 0.1M acetic acid with pH of 4 was used throughout this study. This solution was prepared by adding 6 ml of 1M acetic acid into 1 L distilled water. Then 1M NaOH pellets were added to adjust the pH and calibrated using a pH meter, until it was pH 4. 1 L of 0.1M acetic acid at pH4 was produced and stored at room temperature. Acetic acid was chosen as demineralising solution as it has potential to develop carious lesions at a rapid rate. The PH meter was of the brand Mettler Toledo: in Lab Expert Go-ISM.

### Dosage Ranging Study

Six varnished HAP discs divided into six different groups shown below. The varnished discs where immersed in deionised water for a minimum of 30 minutes before the experiment started, this allowed for water to diffuse into the pores of the HAP disc, for better results.

### Toothpaste Tab Groups:

i) 0% PgGA (control)
ii) 0.5% PgGA
iii) 1.0% PgGA
iv) 0% PgGA with fluoride (control)
v) 0.5% PgGA with fluoride
vi) 1.0% PgGA with fluoride

Each toothpaste tab was dissolved in 20 ml of deionised water, ensuring to continuously stir and wait until the toothpaste tab had completely dissolved and no residue is visually present. This is around 20 seconds up to 1 minute. These solutions were used immediately after dissolving.

A varnished HAP disc is then immersed into the first solution with continual stirring for 2 minutes (to simulate toothbrushing). The HAP disc is removed from this solution and placed in the beaker containing 50 ml of demineralising solution that is maintained at 37° C. (+/−1.0° C.). A small magnetic stirrer was used to constantly agitate the solution to ensure solution was moving over the HAP disc.

The calcium ion concentrations in the solution were monitored using the Ca²⁺ selective electrode and a record was taken every minute over 3 hours. These values were used to calculate the rate of calcium release (RCa²⁺).

The procedure was repeated for each disc in the remaining toothpaste tab groups, respectively.

Three repeats of all the results were completed to ensure the similar results were obtained for accuracy purposes.

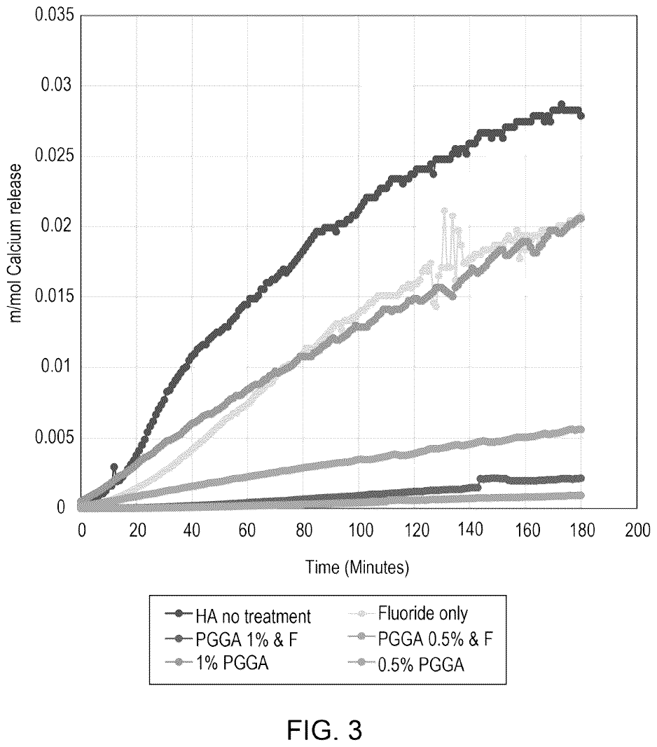

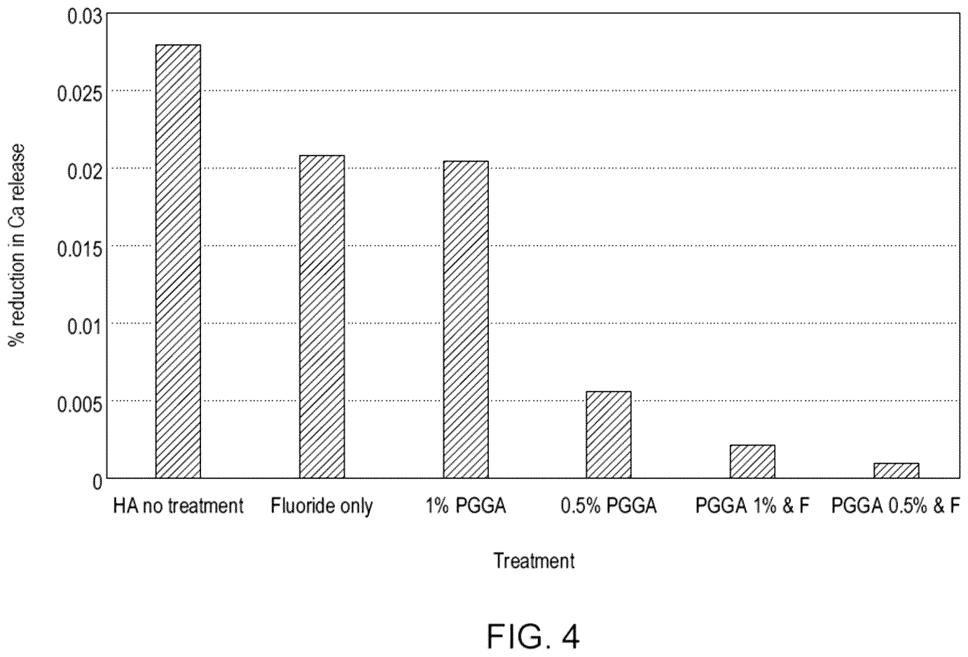

The graphs show that PgGA works synergistically with fluoride on the inhibition of calcium release which is unexpected. This inhibitory effect is as its maximum at 0.5% PgGA with fluoride. 0.5% PgGA with fluoride is a factor of 20× more effective than fluoride alone. Theories of why this has occurred have been discussed but the true reason is unknown. Lower concentrations of PgGA (0.25%) were tested but found to be less effective and not included in the graph (see FIGS. 3-6).

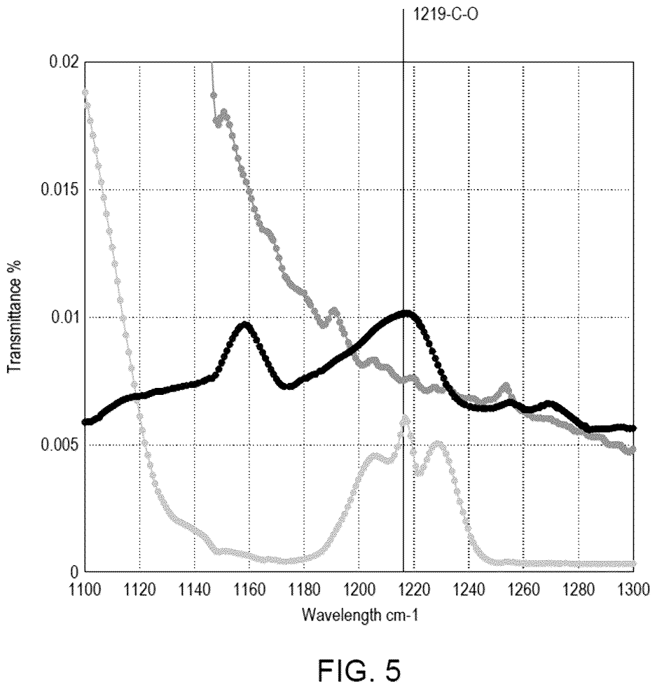

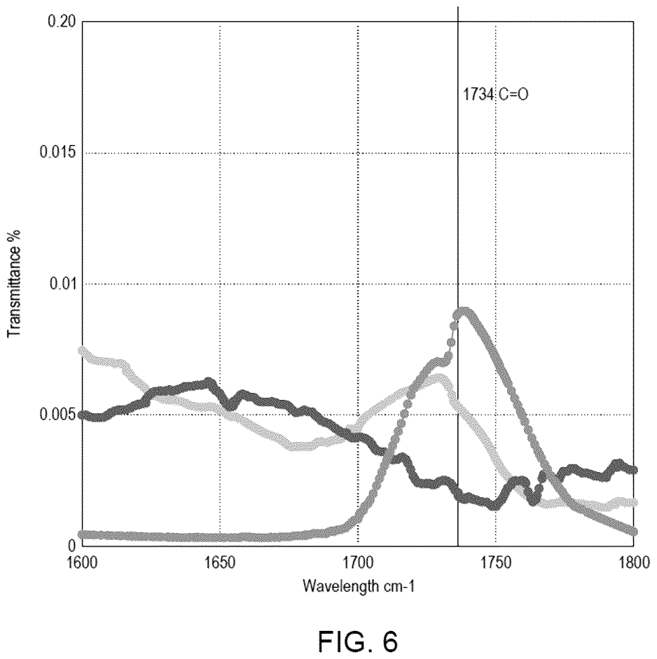

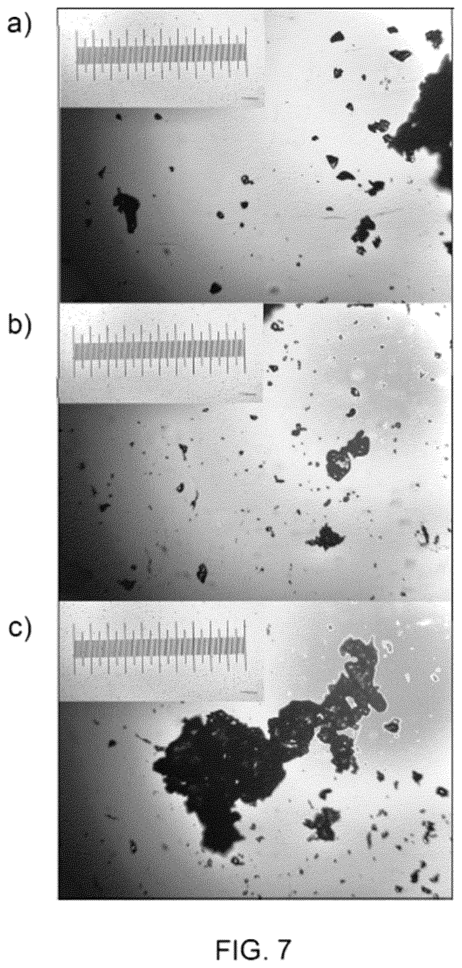

In FIG. 5, we can see a correlation at 1219 C—O and in FIG. 6 we can see a correlation at 1734 C═O. Both these correlations show that PgGA is binding to the HAP surface even after the disc was rinsed. This further supports the results we have seen in the dose ranging.

The FTIR data, shows that PgGA remains even after a through rinse, suggesting that it binds to the tooth enamel surface, preventing tooth decay. We can see correlations in the FTIR data that support this. This is indicative of it having the ability to adhere enamel surface as it has shown inhibition of demineralisation of toothlike material (hydroxyapatite) (FIGS. 5 & 6).

### CITATION LIST

BDJ 2021. 300 million toothpaste tubes go to landfill. British Dental Journal, 230, 390-390.
CHAE, Y., AN, YOUN-JOO., 2016. DEPARTMENT OF ENVIRONMENTAL HEALTH SCIENCE, K. U., 120 NEUNGDONG-RO, GWANGJIN-GU, SEOUL 05029, REPUBLIC OF KOREA., Toxicity and transfer of polyvinylpyrrolidone-coated silver nanowires in an aquatic food chain consisting of algae, water fleas, and zebrafish. Aquatic Toxicology (Amsterdam, Netherlands), 173, 94-104.
CHEN, X., FAIZA G., D. G. GILLAM, R. G. HILL. 2017 “Characterization of casein phosphopeptide-amorphous calcium phosphate based products with and without sodium fluoride.” https://api.semanticscholar.org/CorpusID: 55947741.
DENTALHEALTHORG 2022. “Spit don't rinse” for better oral health.
DUANGTHIP, D., CHU, C 2022. Frontiers | Challenges in Oral Hygiene and Oral Health Policy | Oral Health.
FEATHERSTONE, J. 2008. Dental caries: a dynamic disease process. Australian dental journal, 53.
FRESHSTART 2021. Are Old Toothbrushes & Toothpaste Tubes Recyclable?
JD, F. 1999. Prevention and reversal of dental caries: role of low level fluoride. Community dentistry and oral epidemiology, 27.
KANDUTI, D., STERBENK, P AND ARTNIK, B 2016. FLUORIDE: A REVIEW OF USE AND EFFECTS ON HEALTH.
KIM, E., YANG, J., SUNG, M.-H. & POO, H. 2019. Oral Administration of Poly-Gamma-Glutamic Acid Significantly Enhances the Antitumor Effect of HPV16 E7-Expressing Lactobacillus casei in a TC-1 Mouse Model. Journal of microbiology and biotechnology, 29.
KIZHAKKUMPAT, A., SYED, A., ELGORBAN, A. M., BAHKALI, A, H., KHAN, S 2021. The toxicity analysis of PVP, PVA and PEG surface functionalized ZnO nanoparticles on embryonic as well as adult Danio rerio. Environmental monitoring and assessment, 193.
NAJAR, N. & DAS, S. 2019. POLY-GLUTAMIC ACID (PGA)—STRUCTURE, SYNTHESIS, GENOMIC ORGANIZATION AND ITS APPLICATION: A REVIEW | INTERNATIONAL JOURNAL OF PHARMACEUTICAL SCIENCES AND RESEARCH [Online]. Available: https://ijpsr.com/bft-article/poly-glutamic-acid-pga-structure-synthesis-genomic-organization-and-its-application-a-review/?view=fulltext
NHSUK. 2018a. Fluoride [Online]. @nhsuk. Available: https://www.nhs.uk/conditions/fluoride/
NHSUK. 2018b. Tooth decay [Online]. @nhsuk. Available: https://www.nhs.uk/conditions/tooth-decay/
OGUNLEYE, A., BHAT, A., U. IRORERE, V., HILL, V., WILLIAMS, C. & RADECKA, I. 2015. Poly-γ-glutamic acid: production, properties and applications.
PHE 2022. Local authorities improving oral health.
SAMUELSSON, H. 2014. Is toothpaste an environmental hazard? Master of Science 30 hec, University of Gothenburg.
TOOTHPASTEANDSOCIETY. 2013. Consequences of science, Environmental Impact. Available from: http://chemistryandsociety-toothpaste.weebly.com/environmental-impact.html.

1. A toothpaste composition comprising fluoride and poly gamma glutamic acid or a salt thereof.

2. The toothpaste composition according to claim 1, wherein poly gamma glutamic acid is present as a calcium poly gamma glutamic acid, sodium poly gamma glutamic acid or potassium poly gamma glutamic acid.

3. The toothpaste composition according to claim 1 or claim 2, wherein the poly gamma glutamic acid or salt thereof is present from about 0.1 to about 5 wt %, optionally 0.2 to about 2.5 wt %, optionally from about 0.25 wt % to about 2 wt %, optionally from about 0.5 wt % to about 1 wt %.

4. The toothpaste composition according to any preceding claim, wherein the fluoride is present from about 100 to about 10000 ppm, optionally 200 to about 3000 ppm, optionally from about 500 to about 1700 ppm.

5. The toothpaste composition according to any preceding claim, wherein the poly gamma glutamic acid or salt thereof is present from about 0.25 wt % to about 2 wt % and/or the fluoride is present from about 500 to about 1700 ppm.

6. The toothpaste composition according to any preceding claim, wherein the poly gamma glutamic acid has a molar mass of up to 800 000 g/mol.

7. The toothpaste composition according to any preceding claim, wherein the poly gamma glutamic acid has a molar mass from about 1000 to about 800 000 g/mol, optionally about 5000 to about 700 000 g/mol.

8. The toothpaste composition according to any preceding claim, wherein the poly gamma glutamic acid is present in particular form.

9. The toothpaste composition according to any preceding claim, wherein the poly gamma glutamic acid is present in particular form with a particle size of about 0.1 μm to about 200 μm, optionally about 10 μm to about 50 μm.

10. The toothpaste composition according to any preceding claim, wherein the poly gamma glutamic acid is present in particular form with a particle size distribution in which at least 95% of the particles have a particle size of about 0.1 μm to about 200 μm.

11. The toothpaste composition according to any preceding claim, wherein the poly gamma glutamic acid is present in particular form with a particle size distribution in which at least 80% of the particles have a particle size of about 10 μm to about 50 μm.

12. The toothpaste composition according to any preceding claim, wherein the poly gamma glutamic acid is present in particular form with a particle size distribution in which at least 95% of the particles have a particle size of about 0.1 μm to about 200 μm and at least 80% of the particles have a particle size of about 10 μm to about 50 μm

13. The toothpaste composition according to any preceding claim, wherein said particle size and/or particle size distribution is measured using a measured by light microscope.

14. The toothpaste composition according to any preceding claim, wherein the poly gamma glutamic acid is selected from the group consisting of: a mixture of D and L stereo isomers of glutamic acid, D isomer of glutamic acid and L isomer of glutamic acid.

15. The toothpaste composition according to any preceding claim, wherein the fluoride is selected from the group consisting of: fluoride-containing bioactive glass, fluoride-containing alkalisite glass, sodium fluoride, potassium fluoride, sodium fluoride and sodium monofluorophosphate.

16. The toothpaste composition according to any preceding claim, wherein the toothpaste composition further comprises sodium bicarbonate, potassium bicarbonate, calcium carbonate or a mixture thereof.

17. The toothpaste composition according to claim 16, wherein the toothpaste composition comprises sodium bicarbonate, potassium bicarbonate, calcium carbonate or a mixture thereof in an amount of from about 5 to about 30 wt %, optionally sodium bicarbonate in an amount of from about 5 to about 30 wt %.

18. The toothpaste composition according to any preceding claim, wherein the toothpaste composition further comprises sodium citrate, casein phosphopeptide, potassium nitrate or a mixture thereof.

19. The toothpaste composition according to claim 18, wherein the toothpaste composition comprises sodium citrate, casein phosphopeptide, potassium nitrate or a mixture thereof in an amount of from about 55 to about 85 wt %, optionally sodium citrate in an amount of from about 55 to about 85 wt %.

20. The toothpaste composition according to any preceding claim, wherein the toothpaste composition further comprises sodium methylcocoyl taurate, sodium lauryl sulfate, sodium lauryl sulfoacetate (SLSA), sodium cocoyl glycinate, disodium sodium cocoyl glutamate, sodium cocoyl glutamate, decyl glucoside, lauryl glucoside, betaine or a mixture thereof.

21. The toothpaste composition according to claim 20, wherein the toothpaste composition comprises methylcocoyl taurate, sodium lauryl sulfate, SLSA, sodium cocoyl glycinate, disodium sodium cocoyl glutamate, sodium cocoyl glutamate, decyl glucoside, lauryl glucoside, betaine or a mixture thereof in an amount of from about 0.2 to about 4 wt %, optionally methylcocoyl taurate or sodium lauryl sulfate in an amount of from about 0.2 to about 4 wt %.

22. The toothpaste composition according to any preceding claim, wherein the toothpaste composition comprises one or more additional component(s).

23. The toothpaste composition according to claim 22, wherein the additional component(s) is present from about 3 to about 15 wt %.

24. The toothpaste composition according to any preceding claim, wherein the toothpaste composition comprises:
a. Poly gamma glutamic acid or a salt thereof in an amount of from about 0.25 to about 5 wt %;
b. Fluoride in an amount of from about 500 to about 1700 ppm;
c. Sodium bicarbonate in an amount of from about 5 to about 30 wt %;
d. Sodium citrate in an amount of from about 55 to about 85 wt %;
e. Sodium methylcocoyl taurate and/or sodium lauryl sulfate in an amount of from about 0.2 to about 4 wt %;
f. At least an additional component(s) and wherein the additional component(s) is present in an amount of from about 3 to about 15 wt %;
relative to the total weight of the composition.

25. The toothpaste composition according to any preceding claim, wherein said toothpaste composition is in the form of a paste, powder, tablet or capsule.

26. The toothpaste composition according to any preceding claim, wherein said toothpaste composition is in the form of a tablet.

27. The toothpaste composition according to claim 26, wherein the tablet is a dissolvable tablet.

28. The toothpaste composition according to claim 26 or claim 27, wherein the tablet is a chewable tablet.

29. The toothpaste composition according to any preceding claim, wherein the toothpaste composition is not in the form of a buccal tablet.

30. The toothpaste composition according to any preceding claim, wherein the poly gamma glutamic acid is present in free form.

31. The toothpaste composition according to any preceding claim, wherein the poly gamma glutamic acid is not complexed to another molecule, for example ACP.

32. A method of making a toothpaste tablet, comprising the steps of:
a. providing the toothpaste composition according to any preceding claim; and
b. forming the toothpaste composition into a tablet.

33. The method of making toothpaste tablet according to claim 32, wherein the toothpaste composition is added to a die in step (a), optionally wherein from about 0.1 to about 5 g of the toothpaste composition is added to the die.

34. The method according to claim 32 or claim 33, wherein in step b. a pressure from about 500 to about 1000 psi, optionally from about 650 to about 850 psi, is applied to form the toothpaste composition into a tablet.

35. The method of any one of claims 32 to 34, wherein the tablet is a dissolvable and/or chewable tablet.

36. The method of any one of claims 32 to 35, wherein the tablet is a oral hygiene product.

37. A toothpaste tablet obtained or obtainable according to the method of any one of claims 32 to 36.

38. An oral hygiene product comprising the toothpaste composition according to any one of claims 1 to 31.

39. A method of treating or preventing dental disease, comprising administering the toothpaste composition according to any one of claims 1 to 31, the toothpaste tablet of claim 37 or the oral hygiene product of claim 38 to a subject, optionally wherein said dental disease is selected from the group of cariogenic bacterial diseases and dental caries.

40. The toothpaste composition according to any one of claims 1 to 31 or toothpaste tablet of claim 37 or oral hygiene product according to claim 38 for use in treatment or prevention of dental disease, optionally wherein said dental disease is selected from the group of cariogenic bacterial diseases and dental caries.

41. Use of the toothpaste composition according to any one of claims 1 to 31 or toothpaste table of claim 37 or oral hygiene product according to claim 38 in the manufacture of a medicament for the treatment or prevention of dental disease, optionally wherein said dental disease is selected from the group of cariogenic bacterial diseases and dental caries.
